(19) United States
(12) Patent Application Publication
(10) Pub. No.: US 2025/0065006 A1
(43) Pub. Date: Feb. 27, 2025

(54) DEVICE FOR EVAPORATING VOLATILE SUBSTANCES

(71) Applicant: Zobele Holding, S.p.A, Trento (IT)

(72) Inventors: Luke DI PALMA, Barcelona (ES); Andrea LAGUNA VALER, Barcelona (ES); Alberto José SANCHO MARZO, Barcelona (ES)

(73) Assignee: Zobele Holding, S.p.A, Trento (IT)

(21) Appl. No.: 18/719,288

(22) PCT Filed: Dec. 13, 2022
(86) PCT No.: PCT/EP2022/085564

§ 371 Date: Jun. 13, 2024

(30) Foreign Application Priority Data
Dec. 13, 2021 (ES) P202131146

(51) Int. Cl. A61L 9/12

(52) U.S. Cl. CPC A61L 9/127; A61L 2209/135

(57) ABSTRACT

The device for evaporating volatile substances comprises a container (1) housing a liquid with volatile substances provided with a mouth (2), a closure assembly (5, 6) placed around the mouth (2) of the container (1), a stopper (3) placed in the mouth (2) of the container (1); and wherein at least a portion of the closure assembly (5, 6) is movable between a first and second positions, wherein in the first position, the closure assembly (5, 6) is disengaged from the stopper (3), and in the second position, the closure assembly (5, 6) is engaged to the stopper (3).

It provides a device that allows the container to be filled with a liquid, allowing the wick and the cap to be easily removed and, at the same time, making it safe to use, preventing accidental spillage of the liquid inside the container.

### OBJECT OF THE INVENTION

The present invention relates to a device for evaporating volatile substances, in particular, for evaporating a perfume or insecticide.

### BACKGROUND TO THE INVENTION

Conventional evaporation devices for volatile substances comprise a container in which a liquid containing the volatile substances, such as perfume or insecticide, is housed.

For evaporation, the container usually comprises a wick, which is impregnated in the liquid with the volatile substances and from this wick the volatile substances evaporate into the surroundings when the wick is exposed to air.

To prevent evaporation of the volatile substances when the device is not to be used, a cap, e.g., a screw cap, is placed on the container, which prevents exposure of the wick to the air and thus the evaporation of the liquid with the volatile substances.

Usually, the wick is permanently attached to the container, so that when the liquid inside the container is completely used up, the whole device must be replaced with another one, with the corresponding cost that this represents.

In addition, these devices also comprise a stopper, the function of which is to join the wick to the container. This is usually done in a rather solid way to prevent a child, for example, from being able to dismantle it and drink the liquid.

Therefore, some tools are needed to dismantle it. In addition, as the wick is soiled with fragrance residues, which are oils, handling it is quite dirty and unpleasant.

### DESCRIPTION OF THE INVENTION

Therefore, an objective of the present invention is to provide a device for evaporating volatile substances which allows filling the container with a liquid containing volatile substances, being able to remove the wick and the stopper in a simple way and, at the same time, allowing its use to be safe, preventing accidental spillage of the liquid from the inside of the container.

The device for evaporating volatile substances is described in claim 1, and the dependent claims include optional features of said device.

In particular, the device for evaporating volatile substances according to the present invention comprises:
a container containing a liquid with volatile substances provided with a mouth,
a closure assembly placed around the mouth of the container,
a stopper placed in the mouth of the container; and
   wherein at least a portion of the closure assembly is movable between a first and second positions, wherein:
   in the first position, the closure assembly is disengaged from the stopper, and
   in the second position, the closure assembly is coupled to the stopper.

According to a preferred embodiment, the closure assembly comprises:
a cap detachably placed around the mouth of the container, and
a ring attached to the cap, wherein:
   in the first position, the ring is disengaged from the stopper, and
   in the second position, the ring is engaged to the stopper.

According to a preferred embodiment, the engagement between the ring and the cap is made by means of at least one external projection which, in the first position, is housed in at least one first groove and, in the second position, is housed in at least one second groove.

Preferably, the first and second grooves are arranged at different heights on the cap, in the natural position of use of the cap.

In order to facilitate the movement of the ring actuated by the user, it advantageously comprises a flange.

According to a preferred embodiment, the coupling between the ring and the stopper is achieved by means of at least one internal projection which, in the second position of the ring, is housed in at least one hole of the stopper.

Preferably, the at least one internal projection is positioned on a tab of the ring.

In addition, the at least one hole of the stopper is preferably placed in a skirt extending from the external edge of a disc of the stopper.

According to a preferred embodiment, the cap comprises a beveled rib which, in the second position of the ring, pushes the at least one internal projection into the at least one hole of the stopper.

In addition, the stopper preferably comprises a central hole for a wick, which is in contact at one end with the liquid inside the container and through which the volatile substances evaporate.

According to a preferred embodiment, the at least one external projection is vertically aligned on the ring with the at least one internal projection.

### DESCRIPTION OF THE DRAWINGS

For a better understanding of what has been explained above, drawings are included in which, schematically and only as a non-limiting example, a practical case of embodiment is shown.

FIG. 1 is an exploded perspective view of the device for evaporating volatile substances according to the present invention;

FIG. 2 is an exploded perspective view of the cap, the ring and the stopper of the device for evaporating volatile substances according to the present invention in section;

FIG. 3 is a perspective section view of the relative position between the ring and the cap in the first or closed position;

FIG. 4 is a perspective section view of the relative position between the ring and the cap in the second or removal position; and

FIG. 5 is a section elevation view of a detail of the position of the internal projection of the ring in the first and second, or locking and removal positions.

### DESCRIPTION OF A PREFERRED EMBODIMENT

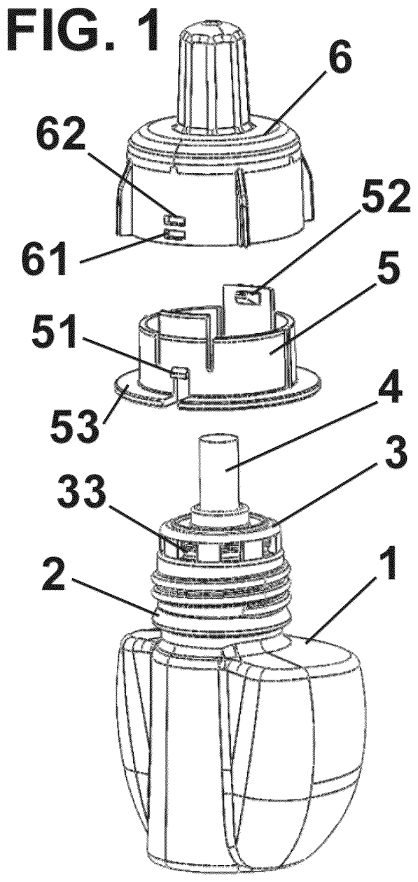

As shown in FIG. 1, the device for evaporating volatile substances according to the present invention comprises a container 1 inside which a liquid containing the volatile substances is housed.

This container 1 comprises a mouth 2, preferably threaded, into which is fitted a stopper 3 provided with a central hole 31 for a wick 4. This wick 4 is in contact at its lower end with the liquid inside the container 1 and volatile substances evaporate from its exposed end.

The evaporation device according to the present invention also comprises a closure assembly formed by a ring 5 and a cap 6.

The ring 5 is movable between two positions: a first or closing position and a second or removal position, as will be explained below.

In addition, the cap 6 blocks the outflow of volatile substances when placed, preferably by screwing, over the mouth 2 of the container 1. For the use of the evaporation device, the cap 6 is removed.

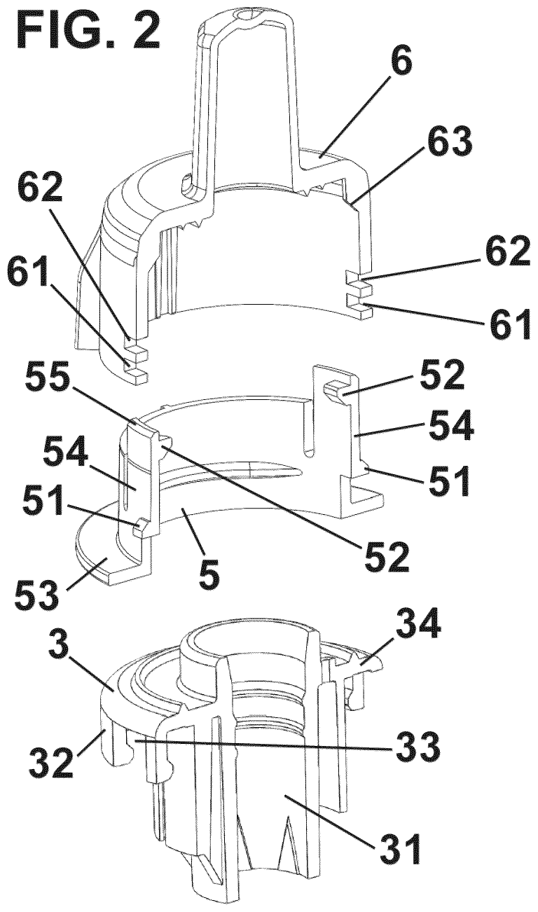

As can be seen, in particular in FIG. 2, the stopper 3, in addition to the central hole 31 defined by a tubular portion for housing the wick 4, comprises a skirt 32 provided with one or more holes 33. This skirt 32 is preferably circular and extends downwards from the edges of a disc 34 of the stopper 3.

In turn, the ring 5 comprises at least one external projection 51 and at least one internal projection 52, and a flange 53, the function of which will be explained below.

As can be seen in FIG. 2, the flange 53 is preferably placed on the lower part of the ring 5, above the flange 53 is arranged the at least one external projection 51 and on the upper part of the ring 5 is arranged the at least one internal projection 52.

Preferably, the internal projection(s) 52 is (are) positioned at the upper end of tabs 54, which can be deformed, as will be explained below. To facilitate the deformation of the tabs 54, the tabs 54 comprise a beveled external edge 55.

The cap 6 comprises at least a first groove 61 and at least a second groove 62, the external projection 51 being accommodated in one or other of the grooves 61, 62, as will be explained below.

Furthermore, the cap 6 comprises a beveled rib 63, preferably extending around the entire internal periphery of the cap 6, which pushes the internal projection(s) 52 into the removal position, as will be explained below.

The evaporation device according to the present invention, with the cap 6 in position, can be placed in two different positions: a closing position and a removal position. With the cap 6 removed it is used for the evaporation of volatile substances.

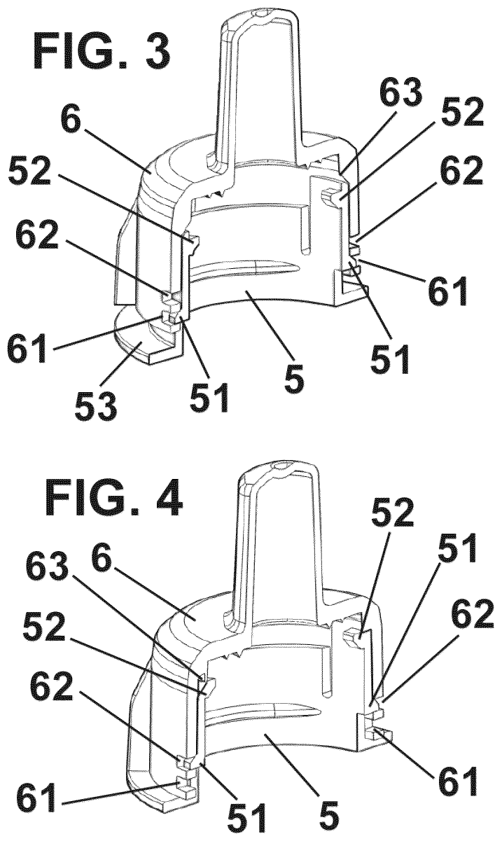

The relative position between the cap 6 and the ring 5 in the closed position is shown in FIG. 3, while in the removed position it is shown in FIG. 4.

In the closed position, the external projections 51 of the ring 5, of which there are two in the embodiment shown, are housed in the first grooves 61 of the cap 6, i.e., in the grooves located below the second grooves 62, in accordance with the embodiment shown.

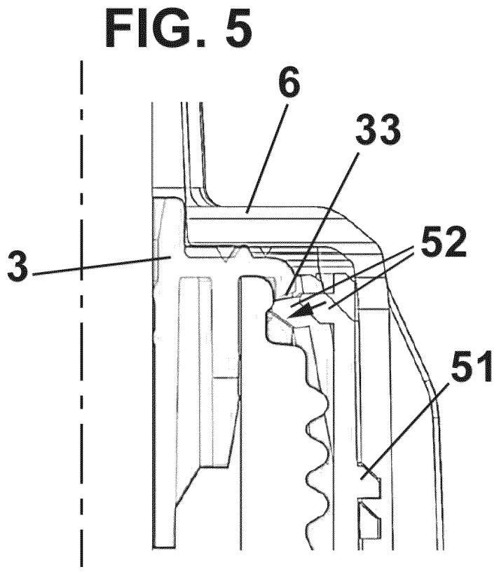

Furthermore, the rib 63 of the cap 6 does not contact the tabs 54 where the internal projections 52 are placed, so that the internal projections 52 are not pushed into the cap 6 and the internal projections 52 are outside the holes 33 of the stopper 3, as shown in the right-hand position of the internal projections 52 in FIG. 5, where they are also shown in the left-hand position with the internal projections 52 in the holes 33 of the stopper 3, in the removal position.

In the closed position, the cap prevents the outflow of volatile substances, and in the position where the device is not used, both before the first use and afterwards, when evaporation of volatile substances is not desired.

To move from this closed position to the removal position, the user pushes the ring 5 upwards with the help of the flange 53.

This thrust will cause the external projections 51 to move out of the first grooves 61 and into the second grooves 62, which are positioned above the first grooves 61.

In the removal position, the rib 63 of the cap pushes the internal projections 52 to be housed into the holes 33, as shown in FIG. 5.

In this removal position, when the user rotates the cap 6 for removal, the ring 5 and the stopper 3 will also be removed, so that the container 1 can be filled with a liquid containing volatile substances and the cap 6, the ring 5 and the stopper 3 can then be placed back into the container 1 to reuse the device in accordance with the present invention.

Although reference has been made to a specific embodiment of the invention, it is obvious to a person skilled in the art that the device described is susceptible to numerous variations and modifications, and that all the details mentioned can be replaced by technically equivalent ones, without departing from the scope of protection defined by the appended claims.

1. A device for evaporating volatile substances, comprising:
a container that houses a liquid with volatile substances provided with a mouth,
a closure assembly placed around the mouth of the container,
a stopper placed in the mouth of the container; and
characterized in that at least a portion of the closure assembly is movable between a first and second positions, wherein:
in the first position, the closure assembly is disengaged from the stopper, and
in the second position, the closure assembly is engaged to the stopper.

2. The device for evaporating volatile substances according to claim 1, wherein the closure assembly comprises:
a cap detachably placed around the mouth of the container, and
a ring engaged to the stopper, wherein:
in the first position, the ring is disengaged from the stopper, and
in the second position, the ring is engaged to the stopper.

3. The device for evaporating volatile substances according to claim 2, wherein the engagement between the ring and the cap is made by means of at least one external projection which, in the first position is housed in at least one first groove and, in the second position, is housed in at least one second groove.

4. The device for evaporating volatile substances according to claim 3, wherein the first and second grooves are arranged at different heights in the cap.

5. The device for evaporating volatile substances according to claim 1, wherein the ring comprises a flange.

6. The device for evaporating volatile substances according to claim 1, wherein the engagement between the ring and the stopper is made by means of at least one internal projection which in the second position of the ring is housed in at least one hole of the stopper.

7. The device for evaporating volatile substances according to claim 6, wherein the at least one internal projection is placed on a tab of the ring.

8. The device for evaporating volatile substances according to claim 6, wherein the at least one hole of the stopper is placed in a skirt extending from the external edge of a disc of the stopper.

9. The device for evaporating volatile substances according to claim 6, wherein the cap comprises a beveled rib which, in the second position of the ring, pushes the at least one internal projection into the at least one hole of the stopper.

10. The device for evaporating volatile substances according to claim 1, wherein the stopper comprises a central hole for a wick.

11. The device for evaporating volatile substances according to claim 3, wherein the at least one external projection is vertically aligned on the ring with the at least one internal projection.
